(19) United States
(12) Patent Application Publication
(10) Pub. No.: US 2005/0064017 A1
(43) Pub. Date: Mar. 24, 2005

(54) Reduction of renal damage in companion animals

(75) Inventor: Tiffany L. Bierer, Vernon, CA (US)

Correspondence Address: FULBRIGHT & JAWORSKI, LLP, 1301 MCKINNEY, SUITE 5100, HOUSTON, TX, 77010-3095, US

(73) Assignee: MARS INCORPORATED, McLean, VA (US)

(21) Appl. No.: 10/891,895

(22) Filed: Jul. 15, 2004

Related U.S. Application Data
(63) Continuation of application No. PCT/US03/02031, filed on Jan. 22, 2003.
Application No. PCT/US03/02031 is a continuation-in-part of application No. 10/264,886, filed on Oct. 4, 2002.
Application No. 10/264,886 is a continuation-in-part of application No. 10/054,093, filed on Jan. 22, 2002.

(51) Int. Cl. A61K 31/202; A23K 1/165; A23K 1/17

(52) U.S. Cl. 424/442; 514/560; 426/635

(57) ABSTRACT

The present invention is directed to a pet food and its use in reducing renal damage in companion animals. More particularly, the pet food maintains muscle mass in animals with renal conditions or animals at risk for developing a renal condition.

This application is a continuation of International Application No. PCT/US03/02031, filed on Jan. 22, 2003, which is a continuation-in-part of application Ser. No. 10/264,886, filed Oct. 4, 2002, which is a continuation-in-part of application Ser. No. 10/054,093, filed Jan. 22, 2002.

### TECHNICAL FIELD

The present invention relates to a pet food for use in reducing renal damage in companion animals. More particularly, the pet food maintains muscle mass in animals with renal conditions or animals at risk for developing a renal condition.

### BACKGROUND OF THE INVENTION

Renal failure is one of the most common causes of death in dogs. In animals which suffer from renal disease, several blood indices are used to determine the severity of the disease. These indices include blood urea nitrogen (BUN) and creatinine. BUN and creatinine levels in the bloodstream increase during the course of renal failure because damage to the kidney of the animal makes the kidney inadequate to filter waste products. Because inadequate filtration of waste products is the fundamental basis of renal disease, BUN and creatinine are considered the primary indicators of renal disease.

A pet food composition that improves several clinical indications (i.e., BUN, creatinine, phosphorous, CO₂ and triglyceride levels) in animals with renal disease is described in U.S. Pat. Nos. 6,039,952 and 6,306,442. This pet food includes from about 10 to about 32% crude protein, from about 8 to about 20% fat, from about 3 to about 25% total dietary fiber, and fermentable fibers which have an organic matter disappearance of 15 to 60% when fermented by fecal bacteria for a 24 hour period, the fibers being present in amounts from about 1 to 11 weight percent of supplemental total dietary fiber. The composition is taught to provide adequate protein, has low phosphorus levels, improves metabolic buffering, and lowers blood triglyceride levels in the animal.

Current dietary therapies to reduce measured BUN, creatinine, and phosphorus levels include decreasing the amount of dietary protein to levels where amino acids are present in insufficient quantities. Decreasing the level of dietary protein decreases BUN since urea is ultimately derived from protein. However, such diets may result in other problems developing for the animal as the animal's protein needs are unmet. Accordingly, there remains a need in this art for a pet food dietary composition which will result in the lowering of BUN, creatinine, and phosphorus levels in the animal without lowering dietary protein to insufficient levels. There also remains a need in this art for a pet food dietary composition which provides improved metabolic buffering and which lowers triglyceride levels in the renal patient.

### BRIEF SUMMARY OF THE INVENTION

The present invention provides a pet food composition for companion animals that suffer from a renal condition or companion animals at risk for developing a renal condition which reduces the level of protein breakdown products handled by the kidneys and thus reduces the stress on the kidneys. The pet food composition of the present invention also helps to maintain muscle mass in the animals with a renal condition or subject to development of a renal condition.

An object of the present invention is a process for reducing renal damage in an animal comprising by feeding the companion animal a pet food product which contains a functional ingredient to modulate renal damage. The pet food product comprises, on a dry matter basis, about 5% to about 40% by weight of protein; about 5% to about 45% by weight of fat; about 0.1% to about 12% by weight of a fiber; about 1% to about 90% by weight carbohydrate, and about 0.1% to about 2% by weight of a functional ingredient. The animal that is fed the pet food product of the present invention either suffers from a renal condition or is at risk for developing a renal condition.

Another object of the present invention is a process for decreasing blood urea nitrogen (BUN) levels of a companion animal comprising the step of feeding to said animal for a time sufficient to decrease BUN levels in the animal. The pet food product comprises, on a dry matter basis, about 5% to about 40% by weight of protein; about 5% to about 45% by weight of fat; about 0.1% to about 12% by weight of a fiber; about 1% to about 90% by weight carbohydrate, and about 0.1% to about 2% by weight of a functional ingredient.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF SUMMARY OF THE DRAWINGS

The following drawings form part of the present specification and are included to further demonstrate certain aspects of the present invention. The invention may be better understood by reference to one or more of these drawings in combination with the detailed description of specific embodiments presented herein:

FIG. 1 is a chart illustrating the blood urea nitrogen (BUN) levels of dogs consuming the inventive diets or control diets over time; and

FIG. 2 is a chart illustrating the blood urea nitrogen (BUN)/creatinine ratio levels in dogs consuming the inventive diets or control diets over time.

### DETAILED DESCRIPTION OF THE INVENTION

A. Definitions

As used herein the specification, “a” or “an” may mean one or more. As used herein in the claim(s), when used in conjunction with the word “comprising”, the words “a” or “an” may mean one or more than one. As used herein “another” may mean at least a second or more.

The term “functional ingredient” as used herein refers to a compound, naturally occurring or synthetic, that is included in a composition and effects a preventative and/or therapeutic response by modulating metabolism in a manner found to be specific to the compound. It is understood that the functional ingredient may include a fat, a carbohydrate, a vitamin, a secondary metabolite, a protein, a prebiotic, a probiotic, or the like, provided the ingredient serves a desired function after consumed by the recipient animal. In a preferred embodiment, the function of the functional ingredient in the subject composition comprises a mitigation of the effect of proteins on the renal system (i.e., kidneys) of the recipient. In other words, the functional ingredient preferably acts to alleviate stress, induced by proteins in the diets of animals suffering from a renal condition or at risk for developing a renal condition.

A compound, component or composition is said to be “acceptable” if its administration can be tolerated by a recipient animal. Such a component is said to be administered in an “effective amount” or “for a time sufficient” if the amount administered is physiologically significant and/or the time the component is administered is physiologically significant. By “physiologically significant,” it is meant that a technical change in the physiology of a recipient animal is observed. For example, the composition that is administered for a time that slows, hinders, delays, completely treats the disease and/or symptoms of a renal condition (e.g., minimizing vitamin, mineral, electrolytes and acid-base imbalances), is considered sufficient. Alternatively, the composition also is capable of maintaining muscle mass in the recipient animal.

A “renal condition” or “renal insufficiency” or “renal disease” or “renal failure” as used herein is defined as the reduced capacity of the kidney to perform its function. Reduced function of the kidneys results in various metabolic abnormalities that modify the nutritional status of the animal. Such abnormalities include, but are not limited to impaired clearance of nitrogenous products of protein metabolism; impaired regulation of sodium, potassium, and phosphorus; impaired vitamin D metabolism; and anorexia. Age of the animal can also play a role in the functional capacity of the kidneys. It is well known to those of skill in the art that as an animal ages, the kidneys are prone to renal conditions.

B. Dietary Therapies for Renal Conditions

Currently, dietary therapies to reduce measured BUN and creatinine levels include decreasing the amount of dietary protein to levels where amino acids are present in insufficient quantities. Decreasing the level of dietary protein decreases BUN since urea is ultimately derived from protein. However, such diets may result in other problems developing for the animal as the animal's protein needs are unmet. The present invention is directed to a dietary system in which the animal is provided a functional ingredient to manage or modulate the levels of protein breakdown products being handled by the kidneys, thus, reducing the stress on the kidneys. In addition to modulating the level of protein breakdown products, the functional ingredient plays a role in maintaining muscle mass in the animal. Thus, the dietary system of the present invention can be fed to an animal that suffers from a renal condition or is subject to development of a renal condition without risk of increasing renal damage and/or decreasing muscle mass.

Dietary protein levels for a healthy animal or animal that does not have a renal condition or is at risk for developing a renal condition are not an issue because healthy diets or maintenance diets do not restrict protein and provide a margin of error to meet the needs of animals of all breeds and sizes. Thus, the dietary protein requirement of a healthy animal is satisfied when the animal's metabolic need for amino acids and nitrogen is satisfied. For example, dietary protein levels for growing dogs are typically 22-25%; dietary protein levels for adult dogs are typically 8-15%; dietary protein levels for growing cats are typically 28-30% ; and dietary protein levels for adult cats are typically greater than 21% (The Merck Index Veterinary Manual, 8^{th} Edition, 1998, pages 1624-1631).

When the animal has compromised kidney function, the levels of protein in the diet become significant. The function of the kidney is to excrete both nitrogenous and non-nitrogenous protein catabolites. The inability or reduced ability of the kidneys to excrete these metabolites results in an accumulation of metabolic by-products in the circulation leading to uremic symptoms and other laboratory abnormalities. Typically, renal failure is treated by reducing the levels of protein in the diet in these animals that are suffering from compromised kidney function. Problems arise from excessive protein restriction in that these low protein diets are associated with protein malnutrition, loss of lean tissue, decreased serum albumin concentrations and other negative side effects. The present invention has developed a pet food composition that provides the necessary levels of protein to meet the metabolic requirements of the animal and maintain lean tissue mass, while modulating the level of circulating catabolites. The functional ingredient in the pet food composition is capable of modulating or decreasing the level of circulating catabolites in an animal that has comprised kidney function or a renal condition.

It is also contemplated in the present invention that the functional ingredient may modulate or decrease the levels of phosphorus. It is well known by those of skill in the art that increased serum phosphorus concentrations can lead to a decrease in the activity of alpha-hydroxylase in the kidney, which contributes to a decrease in the production of calcitriol and an increased production of parathyroid hormone (PTH). PTH is a uremic toxin and is known to contribute to anemia, neurotoxicity, soft tissue calcification and renal osteodystrophy. Thus, a decrease in phosphorus levels can result in a decrease in PTH levels resulting in a decrease in renal failure in the animal.

The pet food product of the present invention generally includes, on a dry matter basis, about 5% to about 40% by weight of protein; about 5% to about 45% by weight of fat; about 0.1% to about 12% by weight of a fiber; about 1% to about 90% by weight carbohydrate, and about 0.1% to about 2% by weight of a functional ingredient. Although it is preferred that the pet food product of be nutritionally complete, a non-nutritionally complete pet food product may also be included in the present invention.

Feeding the renal dietary system of the subject invention to companion animals, such as to a dog and/or to a cat, in which the kidneys are compromised, will help optimize the condition and functional ability of the kidneys of the animal by modulating the by products of metabolism and maintaining lean body mass. Feeding companion animals the pet food of the subject invention will decrease blood urea nitrogen levels (BUN) and blood urea nitrogen (BUN)/creatinine ratio levels, thereby reducing the renal damage to the animal.

Although it is preferred that the pet food product be nutritionally complete, a non-nutritionally complete pet food product may also be included in the present invention. For example, the pet food product may be non-nutritionally complete and used to supplement the basic renal diet. For example, a pet drink or liquid supplement or pet treat comprising the functional ingredient and one or more proteins, carbohydrates, fats, vitamins, minerals or electrolytes can be used as a topper or supplement to a commercially available renal diet.

As is known to one skilled in the art, there are a variety of commonly known pet food products. In the area of cat and dog food, there is wet pet food, semi-moist pet food, dry pet food and pet treats and snacks. Drinks for pets are also available such as milk drinks for cats. Wet pet food generally has a moisture content above about 65%. Semi-moist pet food typically has a moisture content between about 20 to about 65% and can include humectants such as propylene glycol, potassium sorbate, and other ingredients to prevent microbial (i.e., bacteria and mold) growth. Dry pet food (kibble) generally has a moisture content below about 20%, and its processing typically includes extruding, drying and/or baking in heat. Pet treats and snacks can typically be semi-moist chewable treats or snacks; dry treats or snacks in any number of shapes and/or forms; chewable bones; baked, extruded or stamped treats; confection treats/snacks; or other kinds of treats as is known to one skilled in the art. The pet food product may include many different shapes. For example, each shape may comprise the ingredients of the subject composition or, alternatively, a combination of two or more shapes, each having a specific recipe that is cumulative in providing intake of the total composition of the present invention, is contemplated. In a specific example, the pet food product comprises a plurality of kibbles that are characterized by having a shape of either a pea, a bone or a carrot, and each of the shapes comprise a specific recipe. Feeding the pet the combination of one of each shape may provide the composition of the pet food. Alternatively, each shape provides the composition of the pet food and, thus, consumption of any of the shapes provides the animal with comprehensive weight management.

A semi-moist pet food product generally includes ingredients such as cereal grains, meats, fats, vitamins, minerals, water and functional ingredients that are blended together, cooked and packaged. However, any semi-moist pet food formulation known to one skilled in the art can be used. For example, a pet food can be formed by adding, on a dry matter basis, about 5% to about 40% by weight of protein; about 5% to about 45% by weight of fat; about 0.1% to about 12% by weight of a fiber; about 1% to about 90% by weight carbohydrate, and about 0.1% to about 2% by weight of a functional ingredient. Other variations of adding or mixing the ingredients are contemplated and considered within the scope of routine experimentation and processing, and determining such methods as are suitable is within the skill of the ordinary artisan in the art.

In an example of a dry pet food, the ingredients generally include cereal grains, meats, poultry, fats, vitamins, minerals and other functional ingredients. The components are mixed and put through an extruder/cooker. Thereafter, the product is cut or shaped and dried. Non-limiting examples of shapes include a kibble, a pea, a bone, a carrot, a chunk and a pocket. After drying, flavors, fats and other functional ingredients can be coated or sprayed onto the dried product. The spray used is of a kind that is known to one skilled in the art of producing dry pet food.

In an alternative embodiment, an ingredient is specific to a specific shape. For example, if the shape is a carrot, the ingredients may include a carrot powder. Alternatively, if the shape is a pea, the ingredients may include a coloring agent and/or a plant concentrate, such as alfalfa or the like to provide an aesthetic quality.

Although a composition and process for producing a semi-moist pet food product and a dry pet food product has been generally described above, it should be appreciated that any semi-moist pet food composition and process or dry pet food composition and process known to one skilled in the art can be used to produce the pet food product of the subject invention.

A wet pet food and pet treats/snacks are produced as is known to one skilled in the art depending upon the kind of wet pet food product and treat or snack desired. The procedure for preparing the pet food of the present invention depends upon the type of processing required to manufacture the wet pet food or treat/snack. For example, in semi-moist treats/snacks, in which the processing temperature usually does not exceed 5 to 70 degrees C., some or all of the components are added to the treat during processing. However, if the processing temperature of the treat generally exceeds 70 degrees C. and a component such as a functional ingredient displays temperature sensitivity, then the temperature-sensitive component is coated on the finished product.

In specific embodiments, the fat can be provided by a fat that comprises an essential long-chain fatty acid, such as a tetraenoic acid. This is particularly desirable in a pet food product for cats. Functional ingredients includes, but is not limited to a conjugated linoleic acid. Generally, with respect to the pet food product, the functional ingredient acts to mitigate adverse effects of high protein diets. A natural or a synthetic functional ingredient is contemplated. Synthetic and semi-synthetic (i.e., isomerization of vegetable oil using, for example, rumen bacterium Butyrivibrio fibrisolvens) preparations of conjugated linoleic acid have been described and are considered suitable for the present invention (see, for example, U.S. Pat. Nos. 6,410,761; 6,380,409; and 5,554,646, each of which is herein incorporated by reference in their entirety). In such embodiments that the functional ingredient is a conjugated linoleic acid, the amount may be calculated as part of either the functional ingredient content, or of the fat content.

A skilled artisan is aware that a pet food product may further comprise vitamins, trace minerals, enzymes, including those used as processing aids, and flavorings, and is preferred to provide the animal with an entire daily diet to manage renal conditions.

Thus, as described herein, the present invention provides an approach to help optimize the renal or kidney function or capacity in a companion animal. The invention targets a minimization of muscle wasting that can occur from low protein and reduction of renal damage associated with a low to average protein diet. This system reduces the risk of renal damage that is associated with protein by decreasing BUN levels and BUN/creatinine ratio levels in the animal.

C. Example

BUN levels are a direct measure of protein consumption. Dogs fed the HPLC (high protein, low carbohydrate) diet expectedly demonstrated increased BUN levels (FIG. 1) and increased BUN/creatinine ratio levels (FIG. 2). However, the high protein diets comprising conjugated linoleic acid (HPLC+CLA) or a 1,3-diacyglyceride (Econa) maintained normal BUN levels as compared to the Control dieter levels over the 12 week test period. The designations “a”, “b” and “c” indicate significant differences between the designated groups (p>0.05) at the time the blood was taken. Each diet afforded normal BUN levels, with or without the correction for creatinine, for the first half of the testing period. After 6 weeks, dogs fed the HPLC diet demonstrated above normal and significantly different BUN/creatinine ratio levels (FIG. 2) as compared to the CLA, HPLC+CLA and Econa dieters. Similarly, direct measurement of BUN levels yielded above normal and statistically different levels as compared to the CLA, HPLC+CLA and Econa fed dogs (FIG. 1).

This unexpected effect of the inventive diets on protein metabolism indicates that potential medical risks associated with high protein diets are neutralized by co-consumption of the functional ingredients. The reduced risks of renal (e.g. kidney) damage and failure by consumption of the inventive diets is a substantial metabolic improvement in efforts to prevent and treat obese animals.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

1. A process for reducing renal damage in an animal comprising the step of feeding said companion animal a pet food product comprising, on a dry matter basis, about 5% to about 40% by weight of protein; about 5% to about 45% by weight of fat; about 0.1% to about 12% by weight of a fiber; about 1% to about 90% by weight carbohydrate, and about 0.1% to about 2% by weight of a functional ingredient, wherein said functional ingredient reduces renal damage.

2. The process of claim 1, wherein said companion animal is a dog or a cat.

3. The process of claim 1, wherein said companion animal is at risk for developing renal conditions.

4. The process of claim 1, wherein said companion animal suffers from a renal condition.

5. The process of claim 1, wherein said fat comprises an essential long-chain fatty acid.

6. The process of claim 1, wherein said functional ingredient is a conjugated linoleic acid.

7. The process of claim 1, wherein the pet food product is selected from the group consisting of a wet pet food, a semi-moist pet food, a dry pet food, a pet treat, a pet snack, and a pet drink.

8. A pet food for reducing renal damage in an animal comprising, on a dry matter basis, about 5% to about 40% by weight of protein; about 5% to about 45% by weight of fat; about 0.1% to about 12% by weight of a fiber; about 1% to about 90% by weight carbohydrate, and about 0.1% to about 2% by weight of a functional ingredient.

9. The pet food of claim 8, wherein said companion animal is a dog or a cat.

10. The pet food of claim 8, wherein said fat comprises an essential long-chain fatty acid.

11. The pet food of claim 8, wherein said functional ingredient is a conjugated linoleic acid.

12. The pet food of claim 8, wherein the pet food product is selected from the group consisting of a wet pet food, a semi-moist pet food, a dry pet food, a pet treat, a pet snack, and a pet drink.

13. A process for decreasing blood urea nitrogen (BUN) levels of a companion animal comprising the step of feeding to said animal for a time sufficient to decrease BUN levels in the animal, on a dry matter basis, about 5% to about 40% by weight of protein; about 5% to about 45% by weight of fat; about 0.1% to about 12% by weight of a fiber; about 1% to about 90% by weight carbohydrate, and about 0.1% to about 2% by weight of a functional ingredient.

14. The process of claim 12, wherein said animal is a dog.

15. The process of claim 12, wherein said fat comprises essential long-chain fatty acids.

16. The process of claim 12, wherein said functional ingredient is conjugated linoleic acid.

17. The process of claim 12, wherein said animal suffers from a renal condition.

18. The process of claim 12, wherein said animal is at risk for developing a renal condition.

19. The process of claim 12, wherein said animal suffers from a renal condition.
